(19) United States
(12) Patent Application Publication
(10) Pub. No.: US 2025/0366996 A1
(43) Pub. Date: Dec. 4, 2025

(54) Method and Apparatus for Sacroiliac Joint Fusion

(71) Applicant: Tenon Medical, Inc., Los Gatos, CA (US)

(72) Inventors: Wyatt Geist, Melbourne, FL (US); John Souza, Monroe, NC (US)

(21) Appl. No.: 19/298,905

(22) Filed: Aug. 13, 2025

Related U.S. Application Data
(63) Continuation of application No. PCT/US2024/016325, filed on Feb. 17, 2024.
(60) Provisional application No. 63/485,795, filed on Feb. 17, 2023.

(51) Int. Cl. A61F 2/30; A61B 17/00; A61B 17/56; A61B 17/68; A61B 90/00; A61F 2/46

(52) U.S. Cl. CPC A61F 2/30749; A61B 17/68; A61B 90/03; A61F 2/46; A61B 2017/00477; A61B 2017/564; A61B 2017/681; A61B 2090/034; A61F 2002/30156; A61F 2002/30224; A61F 2002/30289; A61F 2002/30331; A61F 2002/30387; A61F 2002/30401; A61F 2002/30481; A61F 2002/30528; A61F 2002/30622; A61F 2002/30995; A61F 2002/4629

(57) ABSTRACT

A surgical fixation assembly and related method of fusing a sacroiliac joint are described herein. The surgical fixation assembly includes a compression member, a stabilizer, and a locking element. The compression member is rotatably implantable into two bone segments separated by a joint. The stabilizer is configured for coupling with the compression member in situ after implantation of the compression member and prevents rotation of the compression member after implantation. The locking element is provided on the stabilizer and is moveable from a first position that allows coupling of the stabilizer and compression member to a second position that prevents uncoupling of the stabilizer from the compression member.

### CROSS-REFERENCES TO RELATED APPLICATIONS

This application is a continuation of International Application No. PCT/US2024/016325, with an international filing date of Feb. 17, 2024, which claims priority to U.S. Provisional Application No. 63/485,795, filed on Feb. 17, 2023 and entitled “SPINAL FIXATION SYSTEM FOR THE SI-JOINT,” the entire content of which is hereby incorporated by reference into this disclosure as if set forth fully herein.

### FIELD OF THE INVENTION

The present disclosure relates generally to orthopedic surgery, and more particularly to implants and methods for sacroiliac joint fusion.

### BACKGROUND OF THE INVENTION

The sacroiliac joint (“SI-JOINT”) is the joint where the sacrum (the triangular bone at the base of the spine) and the ilium (the broad, flat bone that forms the upper part of the hip bone) meet. The sacroiliac joint is a synovial joint cavity filled with synovial fluid, and it is supported by ligaments and muscles. The sacroiliac joint has a sacral component formed by the auricular surfaces of the sacrum, which are ear-shaped surfaces on the sides of the sacrum that articulate with the ilium. The sacral component of the joint is relatively immobile. The iliac component of the joint is formed by the iliac surface of the ilium, which articulates with the sacrum. Between the sacral and iliac components of the sacroiliac joint, there is a fibrous cartilage layer called the interosseous ligament. This ligament connects the two bones and helps to stabilize the joint. The joint is also supported by several other ligaments and muscles, including the sacroiliac ligaments, the iliolumbar ligaments, and the gluteal muscles.

Sacroiliac joint dysfunction is a condition that occurs when there is abnormal motion or alignment of the sacroiliac joint, which is the joint that connects the sacrum (the triangular bone at the bottom of the spine) to the pelvis. This can cause pain in the lower back, buttocks and legs, as well as stiffness and difficulty with mobility.

Sacroiliac joint fusion is a surgical procedure that aims to permanently join the sacrum and ilium bones of the pelvis at the sacroiliac joint. Sacroiliac joint fusion is typically done to treat chronic sacroiliac joint pain, which can result from various conditions, such as degenerative joint disease, traumatic injury, or pregnancy. The procedure involves removing the damaged joint surfaces and replacing them with bone grafts or other materials that promote the growth of new bone across the joint.

One treatment option for sacroiliac joint dysfunction is an implant system. This typically involves the insertion of small implants into the joint to stabilize it and reduce the abnormal motion that is causing the pain and discomfort. The specific type of implant system used will depend on the severity of the dysfunction and the individual patient's needs. Examples of implant systems that may be used include:
SI joint fusion system: This type of implant system involves the fusion of the sacrum and the ilium (the largest bone in the pelvis) using screws and/or plates. This helps to stabilize the joint and prevent abnormal motion. Over time, the bone will grow together, forming a permanent fusion.

SI joint fixation system: This type of implant system involves the use of screws or rods to stabilize the joint, without fusing the bones together. This allows for some degree of motion in the joint but can still help to reduce pain and discomfort.

SI joint distraction system: This type of implant system involves the use of small, minimally-invasive implants to create a distraction force across the joint. This helps to decompress the joint and reduce pain.

In each of these instances, surgical hardware is required to be installed at multiple vertebral levels to achieve a stable fixation or fusion construct. This requires more time in the operating room, more exposure to fluoroscopy, more hardware, increased cost, and an increased need for revision.

There is a need to improve the current fixation and fusion systems and techniques for installation.

### SUMMARY OF THE INVENTION

In some embodiments, the present disclosure describes improved implants and surgery techniques to provide immobilization and stabilization of spinal segments in skeletally mature patients in the treatment of acute and chronic instabilities or deformities including degenerative disc disease, such as spondylolisthesis, injury such as fracture, spinal stenosis, sacroiliac fusion, failed previous fusion. Sacroiliac fusion, including sacroiliac joint dysfunction, is a direct result of sacroiliac joint disruption and degenerative sacroiliitis. In some embodiments, the implant assembly described herein by way of example only allows direct bone grafting by providing lattice topology and fenestration. In some embodiments, the assembly includes anti-rotation stabilization, allowing complete fixation of the sacrum with a single level procedure. In some embodiments, the assembly provides a modular all-in-one system that can be power-driven.

In some embodiments, the surgical fixation assembly of the present disclosure provides improved fixation with several key factors including anti-rotation and stabilization. By way of example, anti-rotation is an important feature because rotation of implanted rotatable (e.g., threaded) devices can be problematic until the device incorporates into the bone (e.g., usually 3 months). By way of example, stabilization is an important feature because of the larger surface area and geometry of two implants (e.g., compression member and stabilizer) becoming one through the joint, it will be more stable, especially compared to threaded cages that taper into the sacrum with a smaller diameter.

In some embodiments, the instant disclosure provides placement methods including reduced steps. In some embodiments, every placement option may be facilitated with power if desired. In some embodiments, the placement methods may provide a user utilizing robotics, surgical navigation, and/or virtual reality that knows the length of the implant before placement the ability to place it with power in one step. In some embodiments, the placement method may include a reverse drill (e.g., for guidepin stabilization), increasing the diameter of the guidepin and including a reverse drill tip on the guidepin to address guidepin impaction, which is during the implant placement, wherein the reverse drill tip will only cut and move forward when spinning counter-clockwise or in reverse with respect to the lead of the implant thread. The implant is preferably inserted spinning in the clockwise direction, while the guidepin is stationary to reduce steps and fluoroscopy shots. Thus, the guidepin may be retracted at the same or similar rate to the insertion of the implant.

In some embodiments, the surgical fixation assembly of the present disclosure comprises an implant made from any suitable biocompatible material, including but not limited to (and by way of example only) titanium, polymer, PEEK, bone, etc. In some embodiments, the surgical fixation assembly of the present disclosure allows for screws or bolts to go through the SI-joint at a predetermined angle using a trajectory guide. In some embodiments, the surgical fixation assembly of the present disclosure includes a radiographic marker to assist the user in visualizing placement of the assembly before, during, and/or after implantation. In some embodiments, the surgical fixation assembly of the present disclosure may be configured for use with navigation, robotics and/or virtual reality systems. In some embodiments, the surgical fixation assembly of the present disclosure may be used with an inserter that allows implants to be placed into the SI space from a lateral trajectory, a posterior-lateral trajectory; and a posterior-medial trajectory.

In some embodiments, the surgical fixation assembly of the present disclosure may be used in a variety of surgical procedures at various locations throughout the body. In some embodiments, the surgical fixation assembly disclosed herein may be optimized for use in a sacroiliac fusion procedure in a spine of a human patient. By way of example, the surgical fixation assembly includes a compression member and a stabilizer configured to be coupled to the compression member in situ (e.g., after the compression member has been implanted in a surgical target site) and secured in place with a locking element.

In some embodiments, the compression member may comprise any device configured to engage two separate bone portions to hold the two separate bone portions in place relative to one another. In some embodiments, the compression member may comprise a bone anchor having a cylindrical elongated shaft with a proximal end, distal end, and a central portion positioned between the proximal and distal ends. In some embodiments, the compression member may further comprise one or more helical threads disposed about the outer surface of the elongated shaft. In some embodiments, the helical threads may have multiple pitch zones configured to gain purchase into different types of bone, for example cortical purchase zones at the proximal and distal ends, and a cancellous pitch zone in the central portion. By way of example, in a sacroiliac fusion procedure, the compression member may be positioned such that the distal end is engaged with the sacrum, the proximal end is engaged with the ilium, and the middle portion spans across the sacroiliac joint.

In some embodiments, the compression member further includes a central lumen extending longitudinally therethrough between the proximal and distal ends. By way of example only, the central lumen may have a generally cylindrical cross-section, and is configured to allow passage of one or more surgical instruments or accessories through the compression member, including but not limited to a guide wire and/or a drill bit.

In some embodiments, the compression member includes one or more coupling features configured to receive at least a portion of the stabilizer to couple the stabilizer to the compression member. In some embodiments, the compression member includes a capture feature to engage with the locking element to prevent uncoupling of the stabilizer from the compression element.

In some embodiments, the stabilizer comprises any device or element configured to couple with the compression member in situ to prevent rotation and/or other movement of the compression member while implanted in bone. In some embodiments, the stabilizer acts as a couplable flange that is associated with the compression member after the compression member has been implanted into bone, that prevents rotation of the compression member by providing a physical barrier with an extended surface area that abuts bone to prevent rotation. In some embodiments, the stabilizer may comprise a secondary fastener that extends through a coupler associated with the compression element and into bone.

In some embodiments, the elongated body may have a cross-section having a triangular shape. In some embodiments, the triangular shape may be oriented such that the apex of the triangle is directed laterally away from the compression member when the stabilizer is coupled to the compression member. By way of example, the elongated body may have any cross-sectional shape capable of providing a sufficient surface area to engage bone. In some embodiments, the stabilizer may have a tapered distal end that facilitates more efficient insertion into bone with minimal disruption. In some embodiments, the stabilizer may have one or more transverse openings configured to allow bony growth through the stabilizer to further secure the positioning of the fusion assembly over time.

By way of example only, the stabilizer includes a coupling feature configured to engage the coupling feature of the compression element to enable a secure and guided coupling of the stabilizer to the compression member.

In some embodiments, stabilizer includes a proximal coupler sized and shaped for coupling with a proximal recess of the compression member. In some embodiments, the proximal coupler may include a locking feature configured to move between a first, unlocked position enabling slideable movement of the stabilizer relative to the compression member in a proximal (e.g., during insertion) and/or distal (e.g., during removal) direction, and a second, locked position preventing movement of the stabilizer relative to the compression member.

In some embodiments, the locking feature may comprise a locking element in the form of a C-clip coupled to the stabilizer within a circumferential groove formed in the proximal coupler. In some embodiments, the locking element may include one or more lateral flanges configured to be received within radial groove(s) of the compression member upon rotation of the locking element from the unlocked position to the locked position. In some embodiments, one or more of the lateral flanges may further include a surface marking providing a visual indication of the orientation and status of the locking element.

In some embodiments, the proximal coupler further includes a threaded aperture configured to receive a portion of an inserter therein.

In some embodiments, the present disclosure describes a method of performing orthopedic fusion surgery, for example a sacroiliac joint fusion in a human spine. In some embodiments, a first step in the method is to establish an operative corridor to the surgical target site, which in this example embodiment is the joint between the ilium and sacrum of a human patient. By way of example only, this fusion procedure can be performed at any level of the sacrum, including but not limited to one or more of S1, S2, and S3 spinal levels. Notably, implantation of the surgical fixation assembly can accomplish sacroiliac fusion with an implant at only one level, however the procedure can be repeated at multiple levels if the surgeon so desires. In any event, the operative corridor may be established by methods commonly known in the surgical arts, including open and/or minimally invasive techniques, with various sub-steps including (but not limited to): (a) determining the access trajectory to the surgical target site (e.g., using robotics or other methods), (b) creating an opening in the patient's skin along the determined access trajectory, (c) dilation of soft tissue between the patient's skin and the surgical target site, and (d) retraction of the soft tissue to maintain the operative corridor and provide better visibility for the surgeon through the dilated opening. In some embodiments, any of sub-steps (a)-(d) may be performed manually or robotically.

In some embodiments, a second step of the method is determining the size (e.g., length and/or diameter) of the compression member to be used in the surgery. In some embodiments, this step may be accomplished using a depth gauge. In some embodiments, this step may be accomplished using robotics.

In some embodiments, a third step of the method is to secure the compression member to a driver instrument, with a drill bit of the driver instrument extending through the central lumen of the compression member and extending distally beyond the distal end of the compression member.

In some embodiments, a fourth step of the method is to insert the compression member through the first bone segment (e.g., the patient's ilium) and into the second bone segment (e.g., the patient's sacrum) such that the proximal end of the compression member is seated within the ilium, the distal end of the compression member is seated within the sacrum, and the central portion of the compression member extends across the sacroiliac joint. By way of example, the compression member may be inserted at any one of the sacral levels SI, S2, or S3 to achieve sacroiliac joint fixation, or at multiple levels if so desired. In some embodiments, the drill bit spins faster than the compression member (e.g., twice as fast). In some embodiments, the drill bit and the compression member rotate in opposite directions (e.g., the drill bit rotates in a clockwise direction while the compression member rotates in a counter-clockwise direction, or the drill bit rotates in a counter-clockwise direction while the compression member rotates in a clockwise direction). In some embodiments, insertion may continue until the drill bit punches through sacrum cortex.

In some embodiments, a fifth step of the method is to remove the driver instrument once the compression member has been seated in a desired position as described above. By way of example, removal of the driver instrument may include sub-steps of decoupling the driver instrument from the compression member and withdrawing the driver instrument from the operative corridor.

In some embodiments, a sixth step of the method includes coupling the stabilizer to the compression member after the compression member has been implanted. In some embodiments, this step of the method may be accomplished by first coupling the stabilizer to an insertion instrument. The stabilizer is then advanced through the operative corridor and coupled with the compression member. As the stabilizer is advanced along the compression member during coupling, the elongated body displaces bone material to either side, which in turn exerts a force back on the elongated body to hold the stabilizer in place, thereby preventing rotation of the compression member.

In practice, it should be noted that occasionally a compression member may be inserted into the patient in a slightly off-target position. Rather than removing and reinserting the compression member, it may be advantageously addressed by selective placement of the stabilizer to extend laterally from the compression member in a manner that overcomes the misaligned implant to achieve a robust fusion without revision. The orientation of the stabilizer may be altered by rotating the compression member to orient the elongated track in the desired direction of stabilizer extension prior to inserting the stabilizer.

In some embodiments, a seventh step of the method is to engage the locking element to prevent backout or uncoupling of the stabilizer from the compression member. For example, once the stabilizer has been fully coupled with the compression element, the locking element may be engaged to secure the coupling of the compression element and stabilizer. To accomplish this, the locking element may be rotated such that one or more lateral flanges are moved from an initial unlocked position in which the lateral flanges are aligned with the elongated body and not positioned under one or more overhangs of the compression member to a locked position in which the lateral flanges are positioned under the overhangs and within radial groove(s). When the lateral flanges are in this second, locked position, the overhangs prevent uncoupling of the stabilizer from the compression member. At this point, the surgical fixation assembly is fully implanted and locked.

In some embodiments, an eighth step of the method comprises removing any remaining instrumentation from the operative corridor and closing the incision.

In some embodiments, the method may include a first step (or pre-step) of inserting a graft member into the sacroiliac joint, for example using a posterior insertion method. In some embodiments, the graft may have holes that help determine the trajectory of incoming implants, for example similar to a femoral nail. The sub-step of determine the access trajectory would then be modified to align the access trajectory with at least one of the holes of the graft. The procedure would then proceed as described herein.

In some embodiments, the compression member may be provided in various shapes and sizes. Even though the overall size of the compression members may vary, most features of the different sized compression members (e.g., the stabilizer engagement features, inserter engagement features, etc.) are the same size to ensure that the same (or identical) stabilizer may be used with any size compression member, which advantageously reduces the cost of, increases the efficiency of, and reduces potential errors associated with providing a kit to users including a variety of sizes of compression members with stabilizers that may be used with any of the compression members.

In some embodiments, the compression member includes a coupling element configured to receive at least a portion of the stabilizer to couple the stabilizer to the compression member. In some embodiments, the coupling element may comprise a cap or washer coupled to the proximal end of the compression member and having at least one lateral flange having an aperture configured to receive at least a portion of the stabilizer therethrough during in situ assembly to capture and hold the stabilizer in a desired angular orientation relative to the compression member.

In some embodiments, the stabilizer comprises as secondary anchor, which may be threadedly engaged with the aperture. In some embodiments, the stabilizer comprises a secondary anchor which passes through the aperture but does not threadedly engage the aperture.

In some embodiments, the compression member includes a coupling feature configured to receive at least a portion of the stabilizer to couple the stabilizer to the compression member. In some embodiments, the coupling feature comprises a guidepost having an elongated shaft configured to extend through the central lumen and a proximal circumferential recess configured to receive at least a portion of the proximal coupler of the stabilizer therein. By way of example, the elongated shaft may have a threaded distal end to enable secure coupling with the stabilizer. In some embodiments, the guidepost may be configured to engage more than one stabilizer.

In some embodiments, the surgical fixation assembly may be used with an alignment bracket, providing an insertion tool for an interbody spacer, as well as a rigid arm that is placed along the inserter shaft before or during the procedure to reduce steps and allow a low-cost alternative to robotics or enable VR and Navigation as a trajectory guide. Multiple drill guide options can be provided with the arm. In some embodiments, the rigid arm includes a proximal end including one or more through-holes in alignment with the interbody spacer to guide the instruments on the determined insertion trajectory.

As additional description to the embodiments described below, the present disclosure describes the following embodiments.

Embodiment 1 is a surgical fixation assembly, comprising a compression member comprising a cylindrical body having a proximal end, a distal end, a middle portion extending longitudinally between the proximal and distal ends, a first bone engaging feature positioned at the proximal end, a second bone engaging feature positioned at the distal end, and a stabilizer engagement feature positioned on an exterior surface of the compression member; an independent stabilizer configured to couple to the compression member by interacting with the stabilizer engagement feature, the stabilizer comprising an elongated body having a third bone engagement feature and a coupling feature; and a locking element rotatably coupled to the stabilizer, the locking element configured for movement between a first position enabling movement of the stabilizer relative to the compression member before or during coupling of the stabilizer and compression member, and a second position preventing movement of the stabilizer relative to the compression member after coupling of the stabilizer and compression member.

Embodiment 2 is the assembly of embodiment 1, wherein the first bone engaging feature comprises a helical thread disposed about the exterior surface of the cylindrical body at the proximal end.

Embodiment 3 is the assembly of embodiments 1 or 2, wherein the second bone engaging feature comprises a helical thread disposed about the exterior surface of the cylindrical body at the distal end.

Embodiment 4 is the assembly of any of embodiments 1 through 3, wherein the stabilizer engagement feature comprises a longitudinal recess formed within the exterior surface of the cylindrical body and extending between the proximal and distal ends.

Embodiment 5 is the assembly of any of embodiments 1 through 4, wherein the longitudinal recess is configured to slideably receive at least a portion of the stabilizer therein.

Embodiment 6 is the assembly of any of embodiments 1 through 5, wherein the compression member has an inner lumen extending longitudinally through the elongated body between openings at the first and second ends.

Embodiment 7 is the assembly of any of embodiments 1 through 6, wherein the compression member includes at least one lateral opening formed within the elongated body.

Embodiment 8 is the assembly of any of embodiments 1 through 7, wherein the stabilizer has a triangular cross-sectional shape.

Embodiment 9 is the assembly of any of embodiments 1 through 8, wherein the stabilizer has at least one transverse opening formed therein.

Embodiment 10 is the assembly of any of embodiments 1 through 9, wherein the locking element has at least one lateral flange configured to engage the compression member when the locking element is in the second position.

Embodiment 11 is a method of fusing a first bone segment and a second bone segment across a joint between the first and second bone segments, comprising the steps of: establishing an operative corridor to a surgical target site; securing a compression member to a driver instrument having an elongated drill bit at a distal end, the compression member comprising a cylindrical body having a proximal end, a distal end, a middle portion extending longitudinally between the proximal and distal ends, a first bone engaging feature positioned at the proximal end, a second bone engaging feature positioned at the distal end, a stabilizer engagement feature positioned on an exterior surface of the compression member, and an inner lumen extending longitudinally through the elongated body between openings at the first and second ends; implanting the compression member into the surgical target site by rotatably advancing the compression member through the first bone segment, across the joint and into the second bone segment until the second bone engaging feature is seated within the second bone segment, the first bone engaging feature is seated within the first bone segment, and the middle portion extends across the joint; advancing an independent stabilizer through the operative corridor toward the implanted compression member; coupling the independent stabilizer in situ to the implanted compression member such that the implanted compression member is prevented from rotating relative to the first and second bone segments; and upon in situ coupling of the stabilizer and the compression member, rotating a locking element coupled with the stabilizer from a first position enabling movement of the stabilizer relative to the compression member, to a second position preventing movement of the stabilizer relative to the compression member.

Embodiment 12 is the method of embodiment 11, wherein the compression member is secured to the driver instrument such that a distal tip portion of the elongated drill bit extends through the inner lumen of the compression member and protrudes distally from the distal end of the compression member.

Embodiment 13 is the method of embodiments 11 or 12, wherein the step of implanting the compression member within the surgical target site includes the sub-steps of: advancing the driver instrument with secured compression member distally through the operative corridor until the distal tip portion of the drill bit contacts the first bone segment; and operating the driver instrument such that the drill bit and compression member rotate.

Embodiment 14 is the method of any of embodiments 11 through 13, wherein prior to performing the step of installing the stabilizer, the driver instrument is detached from the compression member and removed from the operative corridor.

Embodiment 15 is the method of any of embodiments 11 through 14, wherein the first bone engaging feature comprises a helical thread disposed about the exterior surface of the cylindrical body at the proximal end.

Embodiment 16 is the method of any of embodiments 11 through 15, wherein the second bone engaging feature comprises a helical thread disposed about the exterior surface of the cylindrical body at the distal end.

Embodiment 17 is the method of any of embodiments 11 through 16, wherein the step of coupling the independent stabilizer in situ to the implanted compression member comprises the sub-steps of: positioning a portion of a coupling element provided on the stabilizer into a portion of the stabilizer engagement feature of the compression member; and advancing the stabilizer distally along the compression member by slideably translating the coupling element within the stabilizer engagement feature.

Embodiment 18 is the method of any of embodiments 11 through 17, wherein the coupling element comprises an elongated guide rail and the stabilizer engagement feature comprises an elongated track.

Embodiment 19 is the method of any of embodiments 11 through 18, wherein the elongated guide rail is secured within the elongated track with a dovetail feature.

Embodiment 20 is the method of any of embodiments 11 through 19, wherein the locking element has at least one lateral flange configured to engage the compression member when the locking element is in the second position.

Other objectives and advantages of this disclosure will become apparent from the following description taken in conjunction with any accompanying drawings wherein are set forth, by way of illustration and example, certain embodiments of this disclosure. Any drawings contained herein constitute a part of this specification, include exemplary embodiments of the present disclosure, and illustrate various objects and features thereof.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1 is a perspective view of an example of an assembled surgical fixation assembly configured for sacroiliac fusion, according to some embodiments;

FIG. 2 is another perspective view of the surgical fixation assembly of FIG. 1, according to some embodiments;

FIG. 3 is a front plan view of the surgical fixation assembly of FIG. 1, according to some embodiments;

FIG. 4 is a side plan view of the surgical fixation assembly of FIG. 1, according to some embodiments;

FIG. 5 is an exploded perspective view of the surgical fixation assembly of FIG. 1, according to some embodiments;

FIG. 6 is a perspective view of an example of a compression member forming part of the surgical fixation assembly of FIG. 1, according to some embodiments;

FIG. 7 is a front plan view of the compression member of FIG. 6, according to some embodiments;

FIG. 8 is a side plan view of the compression member of FIG. 6, according to some embodiments;

FIG. 9 is a side plan view of the compression member of FIG. 6 rotated 90° from the view of FIG. 8, according to some embodiments;

FIG. 10 is another perspective view of the compression member of FIG. 6, according to some embodiments;

FIG. 11 is a top plan view of the compression member of FIG. 6, according to some embodiments;

FIG. 12 is an exploded top perspective view of an example of a stabilizer and locking element forming part of the surgical fixation assembly of FIG. 1, according to some embodiments;

FIG. 13 is an exploded bottom perspective view of the stabilizer and locking element of FIG. 12, according to some embodiments;

FIG. 14 is a side plan view of the stabilizer and locking element of FIG. 12 coupled together, according to some embodiments;

FIG. 15 is a front plan view of the stabilizer and locking element of FIG. 12 coupled together, according to some embodiments;

FIG. 16 is a top plan view of the stabilizer and locking element of FIG. 12 coupled together, according to some embodiments;

FIGS. 17-19 are perspective views of the surgical fixation assembly of FIG. 1 in various sequential stages of assembly, according to some embodiments;

FIG. 20 is a top plan view of the assembled surgical fixation assembly of FIG. 1 with the locking element in an unlocked position, according to some embodiments;

FIG. 21 is a top plan view of the assembled surgical fixation assembly of FIG. 1 with the locking element in a locked position, according to some embodiments;

FIG. 22 is a flowchart depicting several steps of a method of performing a sacroiliac fusion procedure using the surgical fixation assembly of FIG. 1, according to some embodiments;

FIG. 23 is a perspective view of an example of a robotics platform configured for use with the surgical fixation assembly of FIG. 1, according to some embodiments;

FIG. 24 is a side view of an inserter coupled to a compression member forming part of the surgical fixation assembly of FIG. 1, according to some embodiments;

FIGS. 25-27 are perspective views of a surgical fixation assembly of FIG. 1 during implantation in a sacroiliac fusion procedure, according to some embodiments;

FIG. 28 is a plan view of a fluoroscopic image of a surgical fixation assembly of FIG. 1 after implantation in a sacroiliac fusion procedure, according to some embodiments;

FIGS. 29-32 are front plan, perspective, top plan, and perspective views, respectively, of another example of a compression member forming part of the surgical fixation assembly of FIG. 1, according to some embodiments;

FIG. 33-34 are perspective views of another example of a surgical fixation assembly configured for use in a sacroiliac fusion procedure, according to some embodiments;

FIG. 35-36 are perspective views of another example of a surgical fixation assembly configured for use in a sacroiliac fusion procedure, according to some embodiments;

FIG. 37 is a perspective view of an inserter and alignment fixture constructed to cooperate with the surgical fixation assembly of FIG. 1, according to some embodiments; and

FIG. 38 is a side view of the inserter and alignment bracket of FIG. 37, according to some embodiments.

### DETAILED DESCRIPTION OF THE PREFERRED EMBODIMENT

Illustrative embodiments of the disclosure are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure. The surgical fixation assembly and related methods disclosed herein boasts a variety of inventive features and components that warrant patent protection, both individually and in combination.

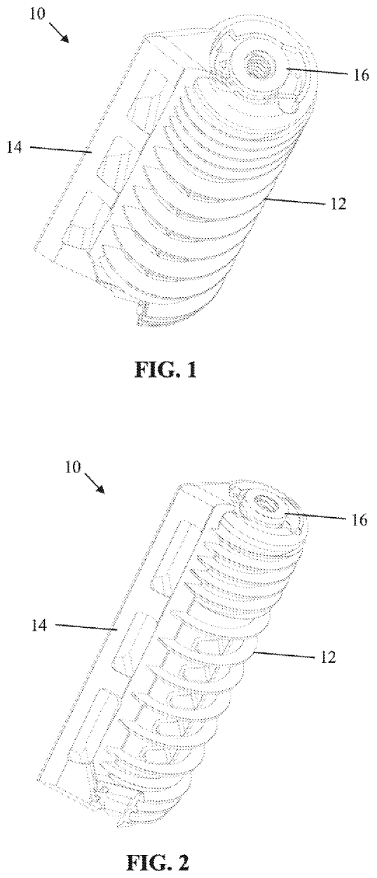

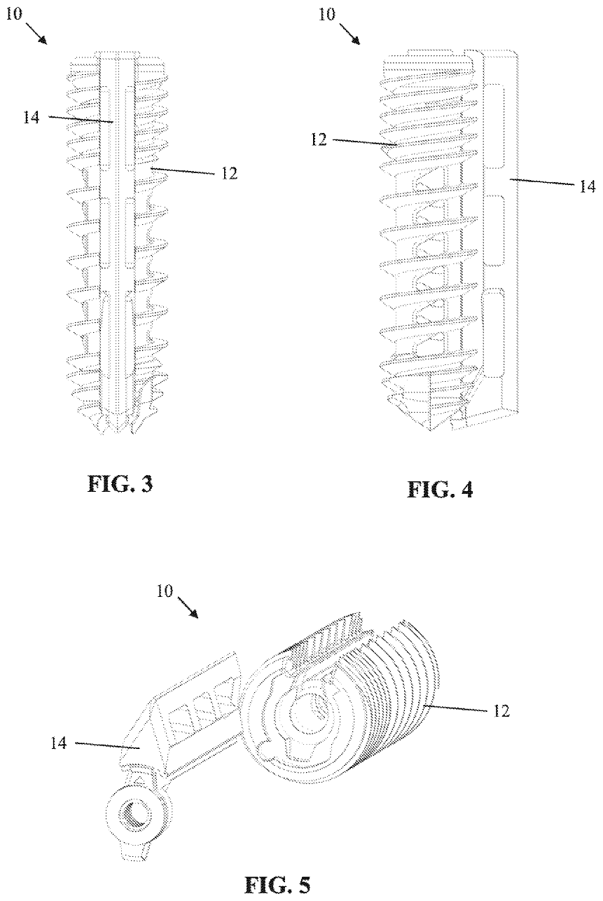

FIGS. 1-5 illustrate an example of a surgical fixation assembly 10 according to some embodiments of the present disclosure. By way of example, the surgical fixation assembly 10 may be used in a variety of surgical procedures at various locations throughout the body. In some embodiments, the surgical fixation assembly 10 disclosed herein may be optimized for use in a sacroiliac fusion procedure in a spine of a human patient. By way of example, the surgical fixation assembly 10 includes a compression member 12 and a stabilizer 14 configured to be coupled to the compression member 12 in situ (e.g., after the compression member 12 has been implanted in a surgical target site) and secured in place with a locking element 16.

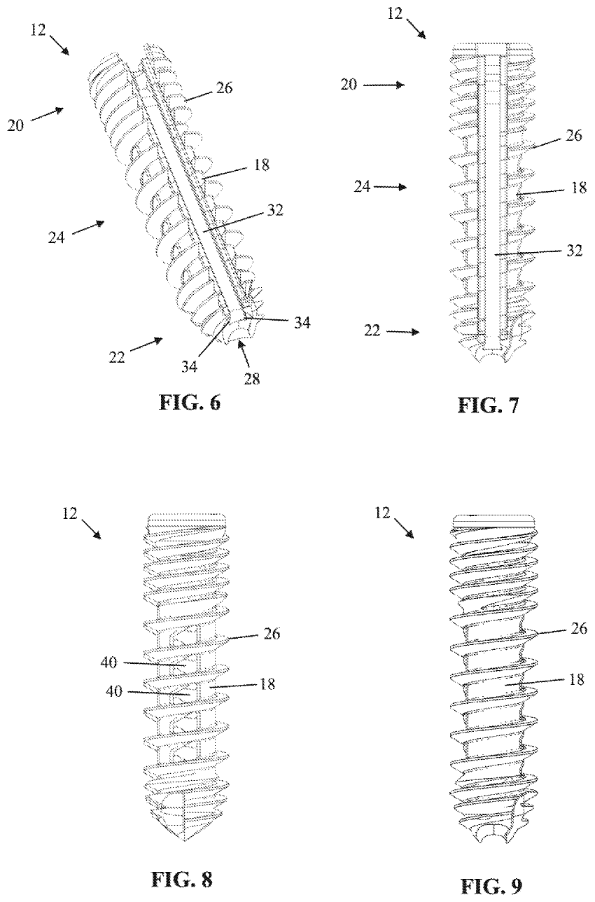

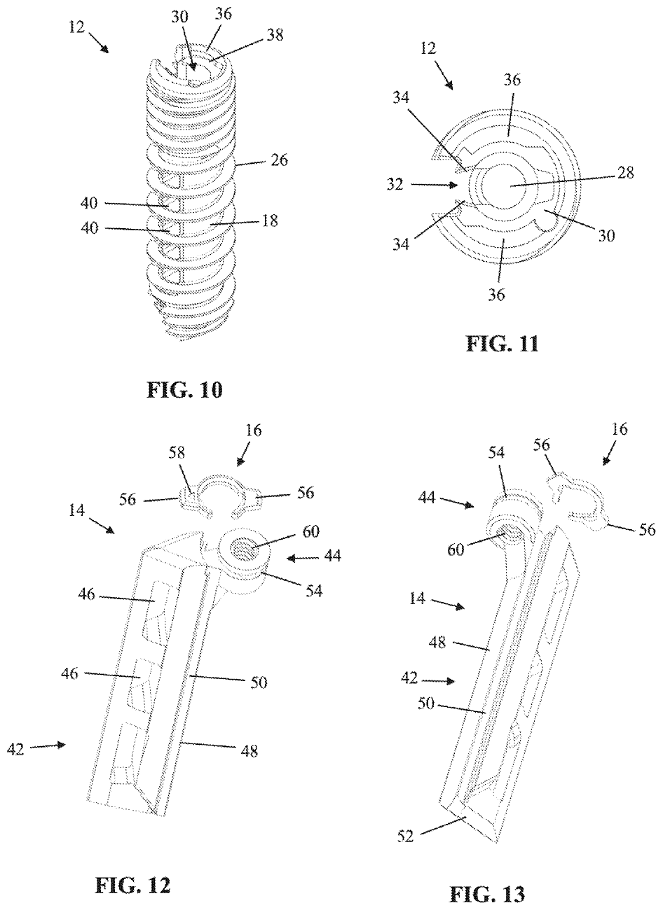

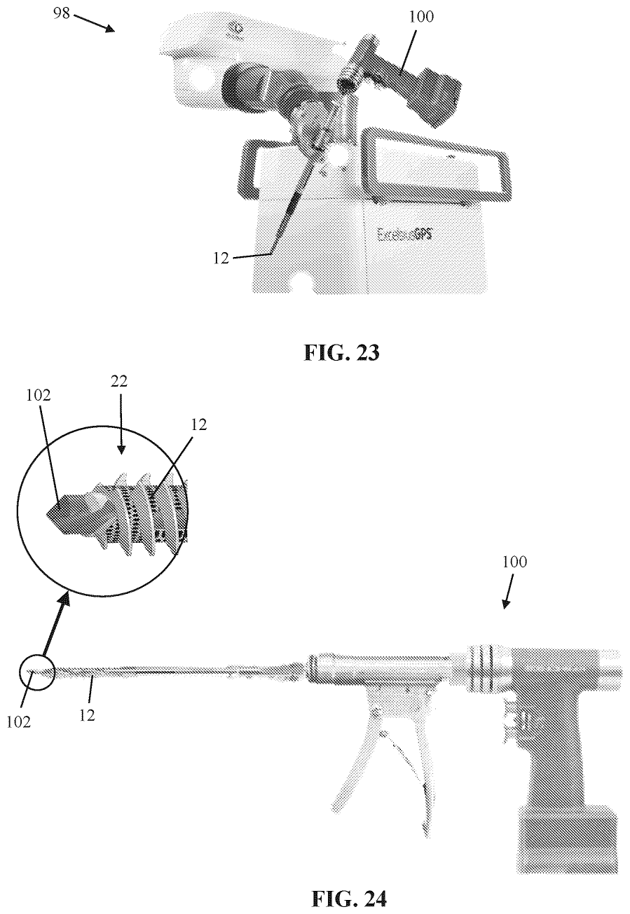

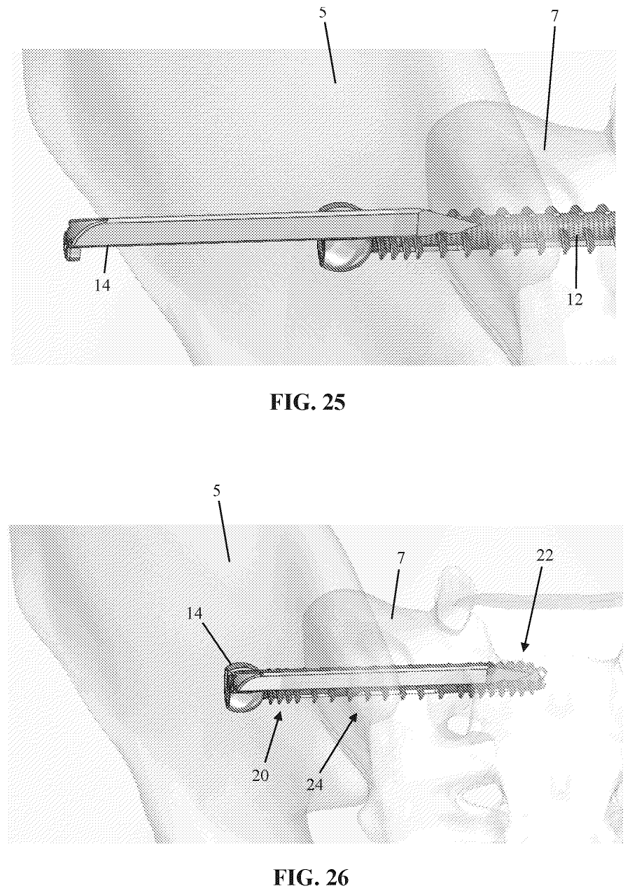

Referring to FIGS. 6-11, the compression member 12 may comprise any device configured to engage two separate bone portions to hold the two separate bone portions in place relative to one another. In some embodiments, the compression member 12 may comprise a bone anchor having a cylindrical elongated shaft 18 with a proximal end 20, distal end 22, and a central portion 24 positioned between the proximal and distal ends 20, 22. In some embodiments, the compression member 12 may further comprise one or more helical threads 26 disposed about the outer surface of the elongated shaft 18. In some embodiments, the helical threads 26 may have multiple pitch zones configured to gain purchase into different types of bone, for example cortical purchase zones at the proximal and distal ends 20, 22, and a cancellous pitch zone in the central portion 24. By way of example, in a sacroiliac fusion procedure, the compression member 12 may be positioned such that the distal end 22 is engaged with the sacrum 5 and the proximal end 20 is engaged with the ilium 7 (See, e.g., FIG. 23).

In some embodiments, the compression member 12 further includes a central lumen 28 extending longitudinally therethrough between the proximal and distal ends 20, 22. By way of example only, the central lumen may have a generally cylindrical cross-section, and is configured to allow passage of one or more surgical instruments or accessories through the compression member, including but not limited to a guide wire and/or a drill bit.

In some embodiments, the compression member 12 includes a coupling feature configured to receive at least a portion of the stabilizer 14 to couple the stabilizer 14 to the compression member 12. For example, the compression member 12 of the instant example embodiment includes a coupling feature comprising a recess 30 formed in the proximal end 20 and configured to receive at least a portion of the proximal coupler 44 of the stabilizer 14 therein, and an elongated track 32 extending longitudinally along the outer surface of the elongated shaft 18 and configured to receive the guide rail 48 of the stabilizer 14 therein during assembly. By way of example, the elongated track 32 may be configured such that the elongated track 32 interrupts the helical thread 26 to enable secure coupling with the stabilizer 14. In some embodiments, the elongated track 32 may have tapered sidewalls 34 to engage the guide rail 48 in a dovetail connection.

In some embodiments, the proximal recess 30 may include one or more overhangs 36 vertically spaced from the proximal recess 30 that create one or more radial grooves 38 between the overhang 36 and the proximal recess 30, the radial grooves 38 configured to receive one or more lateral flanges 56 of the locking element 16 therein to prevent uncoupling of the stabilizer 14 from the compression element 12.

In some embodiments, the elongated shaft 18 may further include one or more lateral openings 40 configured to enable bony fusion through the compression member 12. In some embodiments, the central lumen 28 may be packed with bone growth promoting material after implantation. In some embodiments, the elongated shaft 18 may include a plurality of openings 40 forming a bone growth promoting lattice.

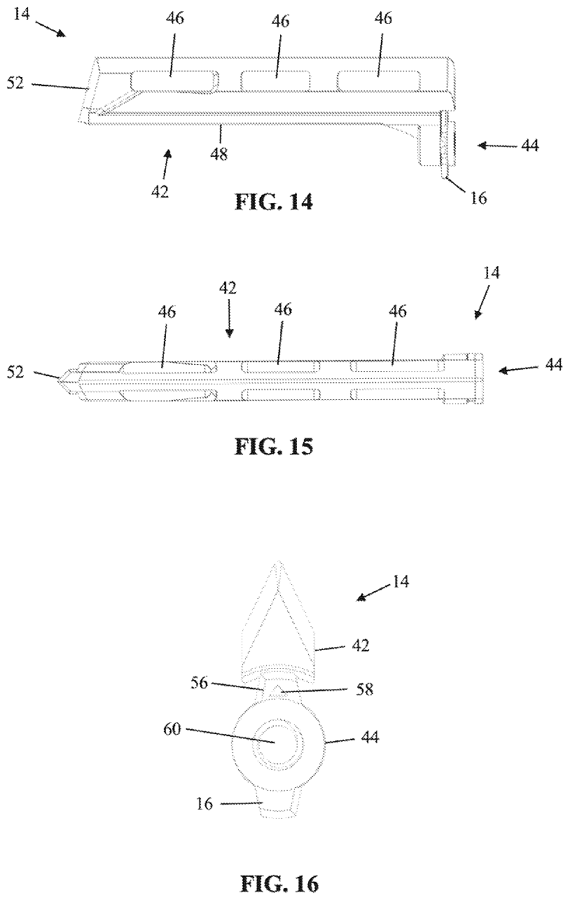

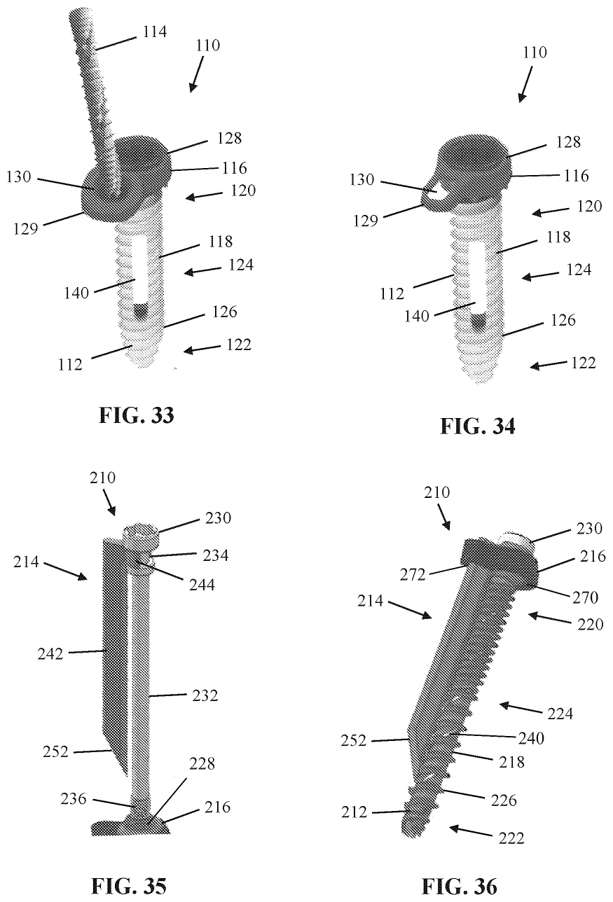

Referring to FIGS. 12-16, in some embodiments, the stabilizer 14 comprises any device or element configured to couple with the compression member 12 in situ to prevent rotation and/or other movement of the compression member 12 while implanted in bone. In some embodiments, the stabilizer 14 acts as a couplable flange that is associated with the compression member 12 after the compression member 12 has been implanted into bone, that prevents rotation of the compression member 12 by providing a physical barrier with an extended surface area that abuts bone to prevent rotation. In some embodiments, the stabilizer may comprise a secondary fastener that extends through a coupler associated with the compression element and into bone (see, e.g., FIGS. 33-34).

In the instant example embodiment, the stabilizer 14 of the comprises an elongated body 42 and a proximal coupler 44. In some embodiments, the elongated body 42 may have a shaped cross-section. In some embodiments, the elongated body 42 may have a cross-section having a triangular shape. In some embodiments, the triangular shape may be oriented such that the apex of the triangle is directed laterally away from the compression member 12 when the stabilizer 14 is coupled to the compression member 12. By way of example, the elongated body 42 may have any cross-sectional shape capable of providing a sufficient surface area to engage bone. In some embodiments, the stabilizer 14 may have a tapered distal end 52 that facilitates more efficient insertion into bone with minimal disruption. In some embodiments, the stabilizer may have one or more transverse openings 46 configured to allow bony growth through the stabilizer to further secure the positioning of the fusion assembly 10 over time.

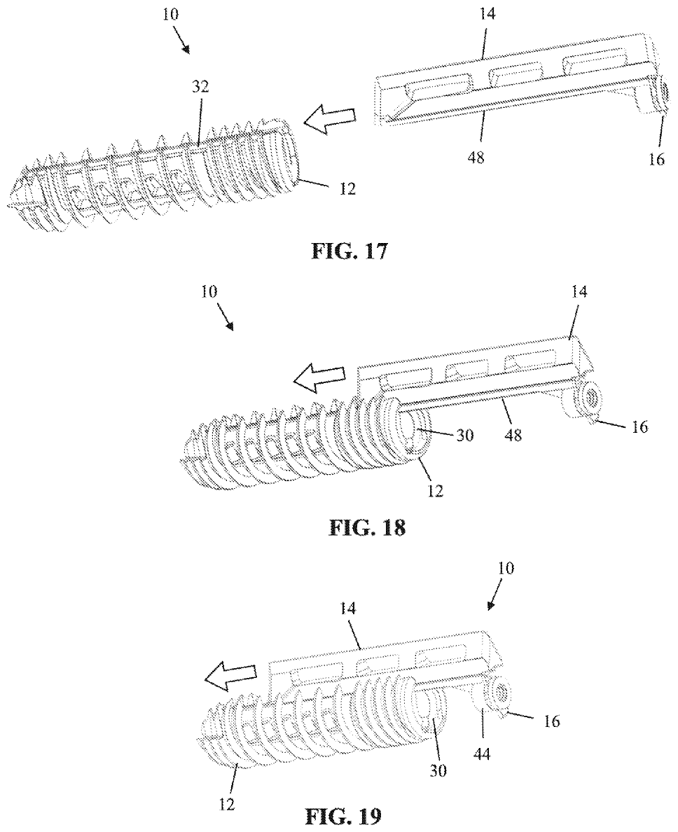

By way of example only, the stabilizer 14 of the present embodiment includes a guide rail 48 configured to slideably engage the elongated track 32 of the compression element 12 described above to enable a secure and guided coupling of the stabilizer 14 to the compression member 12 (as shown for example in FIGS. 17-19). In some embodiments, the guide rail 48 may have tapered sides 50 having a complementary size and taper angle to the tapered sidewalls 34 of the elongated track 32 to create a dovetail engagement between the stabilizer 14 and compression member 12.

In some embodiments, the proximal coupler 44 is sized and shaped for coupling with the proximal recess 30 of the compression member 12. By way of example, the proximal coupler 44 of the instant embodiment has a cylindrical shape, however other shapes are possible. In some embodiments, the proximal coupler 44 may include a locking feature configured to move between a first, unlocked position enabling slideable movement of the stabilizer 14 relative to the compression member 12 in a proximal (e.g., during insertion) and/or distal (e.g., during removal) direction, and a second, locked position preventing movement of the stabilizer 14 relative to the compression member 12.

In some embodiments, the locking feature may comprise a locking element 16 in the form of a C-clip coupled to the stabilizer 14 within a circumferential groove 54 formed in the proximal coupler 44. In some embodiments, the locking element 16 may include one or more lateral flanges 56 configured to be received within the radial groove(s) 38 of the compression member 12 upon rotation of the locking element 16 from the unlocked position to the locked position. In some embodiments, one or more of the lateral flanges 56 may further include a surface marking 58 providing a visual indication of the orientation and status of the locking element 16.

In some embodiments, the proximal coupler 44 further includes a threaded aperture 60 configured to receive a portion of an inserter therein.

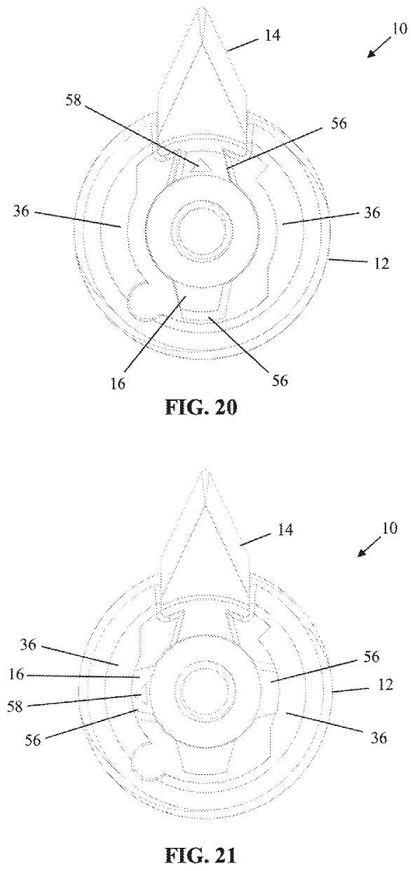

With reference to FIGS. 20-21, once the stabilizer 14 has been fully coupled with the compression element 12, the locking element 16 may be engaged to secure the coupling of the compression element 12 and stabilizer 14. To accomplish this, the locking element 16 may be rotated such that the one or more lateral flanges 56 are moved from an initial unlocked position in which the lateral flanges 56 are aligned with the elongated body 42 and not positioned under the one or more overhangs 36 (see, e.g., FIG. 20) to a locked position in which the lateral flanges 56 are positioned under the overhangs 36 and within the radial groove(s) 38 (see, e.g., FIG. 21). When the lateral flanges 56 are in this second, locked position, the overhangs 36 prevent uncoupling of the stabilizer 14 from the compression member 12. In some embodiments, the stabilizer 14 may be uncoupled from the compression member 12 by returning the locking element 16 to the first, unlocked position, for example by rotating the locking element 16 in the opposite direction until the lateral flanges 56 are clear of the overhang(s) 36.

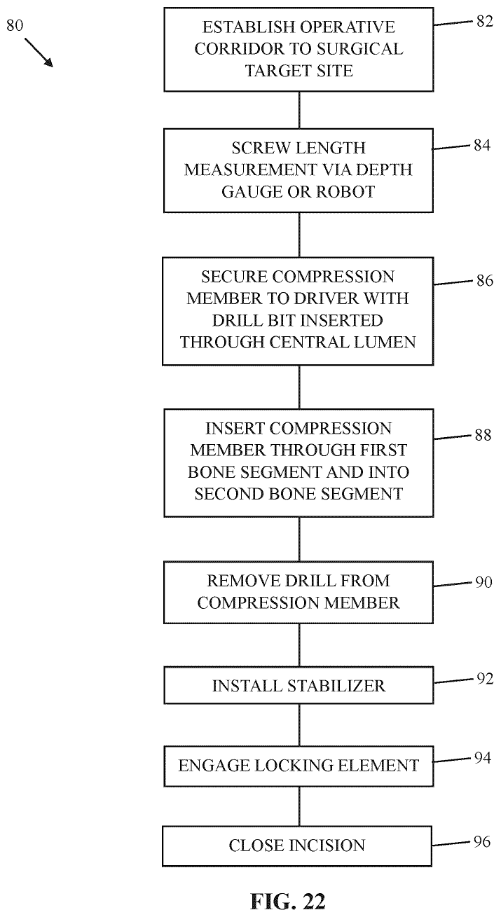

FIG. 22 is a flowchart illustrating several steps in a method 80 of performing orthopedic fusion surgery, for example a sacroiliac joint fusion in a human spine. In some embodiments, a first step 82 in the method 80 is to establish an operative corridor to the surgical target site, which in this example embodiment is the joint between the ilium and sacrum of a human patient. By way of example only, this fusion procedure can be performed at any level of the sacrum, including but not limited to one or more of S1, S2, and S3 spinal levels. Notably, implantation of the surgical fixation assembly 10 can accomplish sacroiliac fusion with an implant at only one level, however the procedure can be repeated at multiple levels if the surgeon so desires. In any event, the operative corridor may be established by methods commonly known in the surgical arts, including open and/or minimally invasive techniques, with various sub-steps including (but not limited to): (a) determining the access trajectory to the surgical target site (e.g., using robotics or other methods), (b) creating an opening in the patient's skin along the determined access trajectory, (c) dilation of soft tissue between the patient's skin and the surgical target site, and (d) retraction of the soft tissue to maintain the operative corridor and provide better visibility for the surgeon through the dilated opening. In some embodiments, any of sub-steps (a)-(d) may be performed manually or robotically, for example using a robotics platform 98 like the one shown in FIG. 23.

In some embodiments, a second step 84 of the method 80 is determining the size (e.g., length and/or diameter) of the compression member 12 to be used in the surgery. In some embodiments, this step may be accomplished using a depth gauge. In some embodiments, this step may be accomplished using robotics.

In some embodiments, a third step 86 of the method 80 is to secure the compression member 12 to a driver instrument 100, with a drill bit 102 of the driver instrument 100 extending through the central lumen 30 of the compression member 12 and extending distally beyond the distal end 22 of the compression member 12, as shown by way of example only in FIG. 24. In some embodiments, the compression member 12 may be secured to the driver instrument 100 by way of a threaded engagement between the proximal end 20 of the compression member 12 and the driver instrument 100.

In some embodiments, a fourth step 88 of the method 80 is to insert the compression member 12 through the first bone segment (e.g., the patient's ilium 5) and into the second bone segment (e.g., the patient's sacrum 7) such that the proximal end 20 of the compression member 12 is seated within the ilium 5, the distal end 22 of the compression member 12 is seated within the sacrum 7, and the central portion 24 of the compression member 12 extends across the sacroiliac joint 9, as shown by way of example in FIGS. 26 and 28. By way of example, the compression member 12 may be inserted at any one of the sacral levels S1, S2, or S3 to achieve sacroiliac joint fixation, or at multiple levels if so desired. In some embodiments, the drill bit 102 spins faster than the compression member 12 (e.g., twice as fast). In some embodiments, the drill bit 102 and the compression member 12 rotate in opposite directions (e.g., the drill bit 102 rotates in a clockwise direction while the compression member 12 rotates in a counter-clockwise direction, or the drill bit 102 rotates in a counter-clockwise direction while the compression member 12 rotates in a clockwise direction). In some embodiments, insertion may continue until the drill bit 102 punches through sacrum cortex.

In some embodiments, a fifth step 90 of the method 80 is to remove the driver instrument 100 once the compression member 12 has been seated in a desired position as described above. By way of example, removal of the driver instrument 100 may include a sub-steps of decoupling the driver instrument from the compression member 12 and withdrawing the driver instrument 100 from the operative corridor.

In some embodiments, a sixth step 92 of the method 80 includes coupling the stabilizer 14 to the compression member 12 after the compression member 12 has been implanted as described herein. In some embodiments, this step 92 of the method 80 may be accomplished by first coupling the stabilizer 14 to an insertion instrument, for example by threadedly engaging an insertion instrument (not shown) with the threaded aperture 60 of the stabilizer 14. The stabilizer 14 is then advanced through the operative corridor and coupled with the compression member 12 by advancing the guide rail 48 of the stabilizer 14 distally along the elongated track 32 of the compression member 12 until the proximal coupler 44 of the stabilizer 14 is seated with in the proximal recess 30 of the compression member 12. As the stabilizer 14 is advanced along the compression member 12, the elongated body 42 displaces bone material to either side, which in turn exerts a force back on the elongated body 42 to hold the stabilizer 14 in place, thereby preventing rotation of the compression member 12.

In practice, it should be noted that occasionally a compression member 12 may be inserted into the patient in a slightly off-target position. Rather than removing and reinserting the compression member 12, it may be advantageously addressed by selective placement of the stabilizer 14 to extend laterally from the compression member 12 in a manner that overcomes the misaligned implant to achieve a robust fusion without revision. The orientation of the stabilizer 14 may be altered by rotating the compression member 12 to orient the elongated track 32 in the desired direction of stabilizer 14 extension prior to inserting the stabilizer 14.

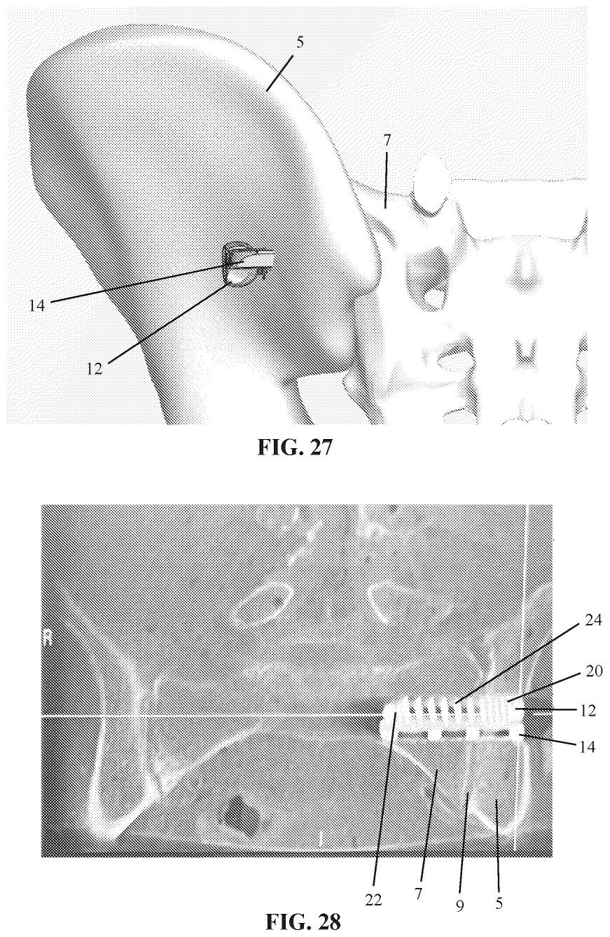

In some embodiments, a seventh step 94 of the method 80 is to engage the locking element 16 to prevent backout or uncoupling of the stabilizer 14 from the compression member 12. As described above with reference to FIGS. 20-21, once the stabilizer 14 has been fully coupled with the compression element 12, the locking element 16 may be engaged to secure the coupling of the compression element 12 and stabilizer 14. To accomplish this, the locking element 16 may be rotated such that the one or more lateral flanges 56 are moved from an initial unlocked position in which the lateral flanges 56 are aligned with the elongated body 42 and not positioned under the one or more overhangs 36 (see, e.g., FIG. 20) to a locked position in which the lateral flanges 56 are positioned under the overhangs 36 and within the radial groove(s) 38 (see, e.g., FIG. 21). When the lateral flanges 56 are in this second, locked position, the overhangs 36 prevent uncoupling of the stabilizer 14 from the compression member 12. At this point, the surgical fixation assembly 10 is fully implanted and locked, as shown by way of example in FIGS. 26-28.

In some embodiments, an eighth step 96 of the method comprises removing any remaining instrumentation from the operative corridor and closing the incision.

In some embodiments, the method 80 may include a first step (or pre-step) of inserting a graft member into the sacroiliac joint, for example using a posterior insertion method. In some embodiments, the graft may have holes that help determine the trajectory of incoming implants, for example similar to a femoral nail. The sub-step of determine the access trajectory would then be modified to align the access trajectory with at least one of the holes of the graft. The procedure would then proceed as described herein.

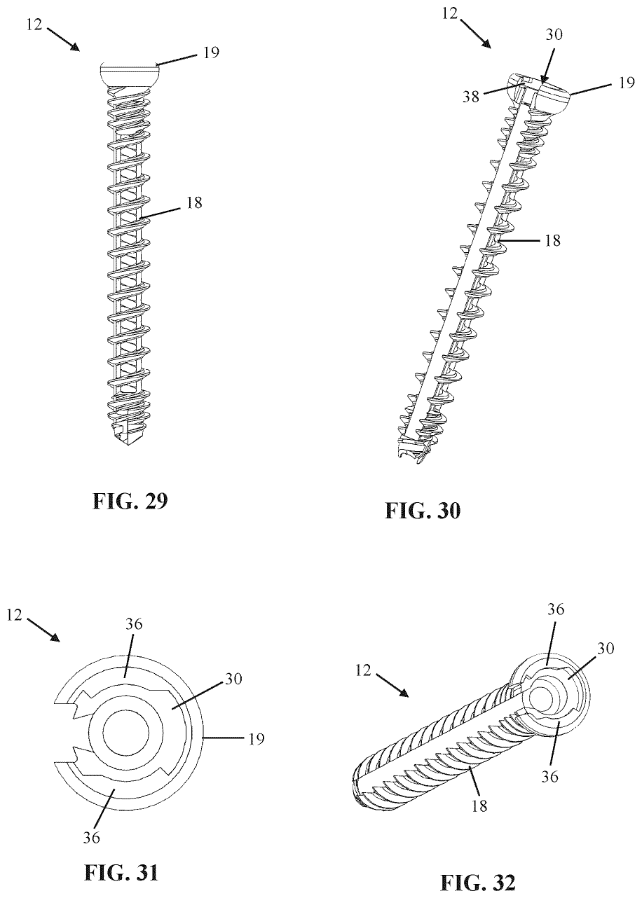

In some embodiments, the compression member 12 may be provided in various shapes and sizes. For example, FIGS. 29-32 illustrate a compression member 12 with an elongated shaft 18 having an increased length and smaller diameter than the compression member 12 described previously. Most features of the compression member 12 shown in FIGS. 29-32 are the same as those described above with the exception of a more pronounced head 19, which is necessary to provide the proximal recess 30, overhang 36, and radial groove 38 having the same size dimensions of the previously described compression member 12. This ensures that the same (or identical) stabilizer 14 may be used with any size compression member 12, which advantageously reduces the cost of, increases the efficiency of, and reduces potential errors associated with providing a kit to users including a variety of sizes of compression members 12 with stabilizers 14 that may be used with any of the compression members 12.

FIGS. 33-34 illustrate a surgical fixation implant 110 according to another embodiment of the disclosure. By way of example only, the surgical fixation implant 110 includes a compression member 112, a stabilizer 114, and a coupling element 116. In some embodiments, the compression member 112 may comprise a bone anchor having a cylindrical elongated shaft 118 with a proximal end 120, distal end 122, and a central portion 124 positioned between the proximal and distal ends 120, 122. In some embodiments, the compression member 112 may further comprise one or more helical threads 126 disposed about the outer surface of the elongated shaft 118. By way of example, in a sacroiliac fusion procedure, the compression member 112 may be positioned such that the distal end 122 is engaged with the sacrum 5 and the proximal end 120 is engaged with the ilium 7, as described above.

In some embodiments, the compression member 112 includes a coupling element 116 configured to receive at least a portion of the stabilizer 114 to couple the stabilizer 114 to the compression member 112. For example, the coupling element 116 of the instant example embodiment comprises a cap or washer 128 coupled to the proximal end 120 of the compression member 112 and having a lateral flange 129 having an aperture 130 configured to receive at least a portion of the stabilizer 114 therethrough during in situ assembly to capture and hold the stabilizer 114 in a desired angular orientation relative to the compression member 112. In some embodiments, the angular orientation of the stabilizer 114 relative to the compression member 112 may be with a range of 0° to 30°. In some embodiments, the angular orientation of the stabilizer 114 relative to the compression member 112 is about 22°. In embodiments, the angular orientation of the stabilizer 114 relative to the compression member 112 may have an extreme angulation of about 28°. In some embodiments, the cap or washer 128 may have a “FIG. 8” shape. In some embodiments, the elongated shaft 118 may further include one or more lateral openings 140 configured to enable bony fusion through the compression member 112.

In some embodiments, the stabilizer 114 comprises as secondary anchor, which may be threadedly engaged with the aperture 130. In some embodiments, the stabilizer 114 comprises a secondary anchor which passes through the aperture 130 but does not threadedly engage the aperture 130.

FIGS. 35-36 illustrate an example of a surgical fixation implant 210 according to some embodiments of the present disclosure. By way of example, the surgical fixation implant 210 may be used in a variety of surgical procedures at various locations throughout the body. In some embodiments, the surgical fixation implant 210 disclosed herein may be optimized for use in a sacroiliac fusion procedure in a spine of a human patient. By way of example, the surgical fixation implant 210 includes a compression member 212, a stabilizer 214 configured to be coupled to the compression member 212 in situ (e.g., after the compression member 212 has been implanted in a surgical target site), and secured in place by way of a guide cap or washer 216.

In some embodiments, the compression member 212 may comprise any device configured to engage two separate bone portions to hold the two separate bone portions in place relative to one another. In some embodiments, the compression member 212 may comprise a bone anchor having a cylindrical elongated shaft 218 with a proximal end 220, distal end 222, and a central portion 224 positioned between the proximal and distal ends 220, 222. In some embodiments, the compression member 212 may further comprise one or more helical threads 226 disposed about the outer surface of the elongated shaft 218. In some embodiments, the helical threads 226 may have multiple pitch zones configured to gain purchase into different types of bone, for example cortical purchase zone at the proximal end 220 and a cancellous pitch zone at the distal end 222. By way of example, in a sacroiliac fusion procedure, the compression member 12 may be positioned such that the distal end 222 is engaged with the sacrum 5 and the proximal end 220 is engaged with the ilium 7 as described above.

In some embodiments, the compression member 212 further includes a central lumen 228 extending longitudinally therethrough between the proximal and distal ends 220, 222. By way of example only, the central lumen 228 may have a generally cylindrical cross-section and is configured to allow passage of one or more surgical instruments or accessories through the compression member, including but not limited to a guide wire and/or a drill bit and/or guidepost 230 for the stabilizer 214.

In some embodiments, the compression member 212 includes a coupling feature configured to receive at least a portion of the stabilizer 214 to couple the stabilizer 214 to the compression member 212. For example, the compression member 212 of the instant example embodiment includes a coupling feature comprising a guidepost 230 having an elongated shaft 232 configured to extend through the central lumen 228 and a proximal circumferential recess 234 configured to receive at least a portion of the proximal coupler 244 of the stabilizer 214 therein. By way of example, the elongated shaft 232 may have a threaded distal end 236 to enable secure coupling with the stabilizer 214.

In some embodiments, the elongated shaft 218 may further include one or more lateral openings 240 configured to enable bony fusion through the compression member 212. In some embodiments, the elongated shaft 218 may include a plurality of openings 240 forming a bone growth promoting lattice.

In some embodiments, the stabilizer 214 comprises any device or element configured to couple with the compression member 212 in situ to prevent rotation and/or other movement of the compression member 212 while implanted in bone. In some embodiments, the stabilizer 214 acts as a couplable flange that is associated with the compression member 212 after the compression member 212 has been implanted into bone, that prevents rotation of the compression member 212 by providing a physical barrier with an extended surface area that abuts bone to prevent rotation.

In the instant example embodiment, the stabilizer 214 comprises an elongated body 242 and a proximal coupler 244. In some embodiments, the elongated body 242 may have a shaped cross-section. In some embodiments, the elongated body 242 may have a cross-section having a triangular shape. In some embodiments, the triangular shape may be oriented such that the apex of the triangle is directed laterally away from the compression member 212 when the stabilizer 214 is coupled to the compression member 212. By way of example, the elongated body 242 may have any cross-sectional shape capable of providing a sufficient surface area to engage bone. In some embodiments, the stabilizer 214 may have a tapered distal end 252 that facilitates more efficient insertion into bone with minimal disruption.

In some embodiments, the proximal coupler 244 is sized and shaped for coupling with the circumferential recess 234 of the guidepost 230. In some embodiments, the guidepost 230 extends through a first aperture 270 of the guide cap or washer 216 and into the central lumen 228 and threadedly engages a distal aspect of the central lumen 228, while the elongated body 242 of the stabilizer 214 extends through a second aperture 272 laterally offset from the first aperture 270. In this manner, the stabilizer 214 is secured to the compression member 212.

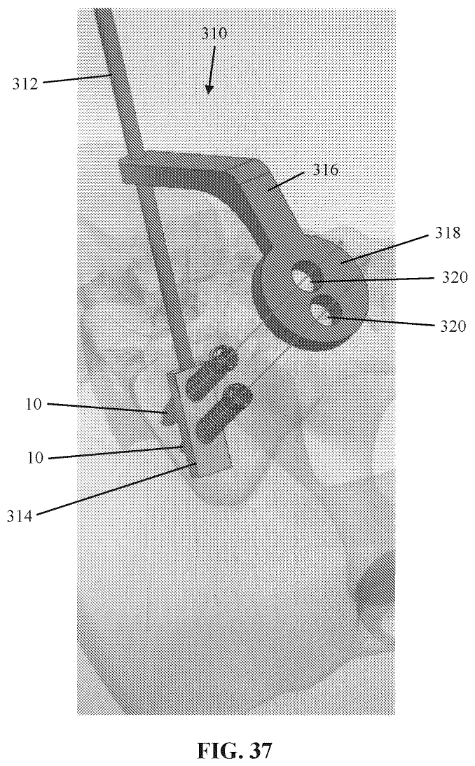

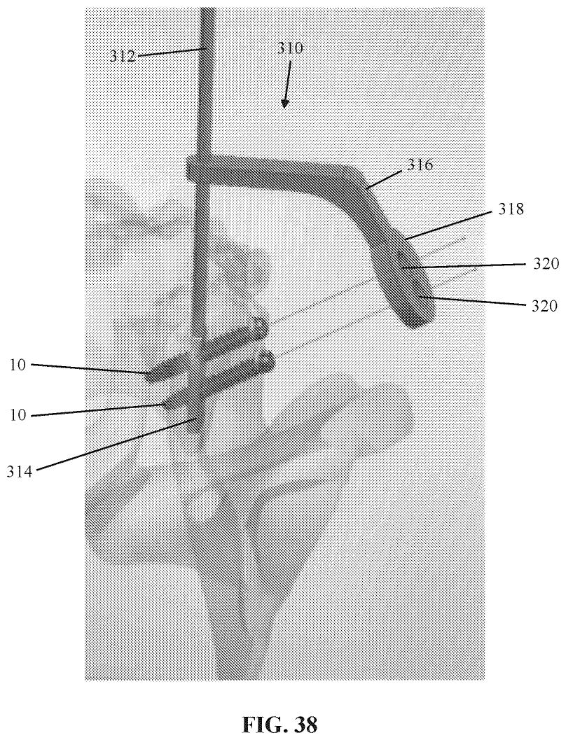

FIGS. 37-38 illustrate an example of an alignment bracket 310 configured for use with the surgical fixation assembly 10 and associated instrumentation described herein, providing an insertion tool 312 for an interbody spacer 314, as well as a rigid arm 316 that is placed along the inserter shaft 312 before or during the procedure to reduce steps and allow a low-cost alternative to robotics or enable VR and Navigation as a trajectory guide. Multiple drill guide options can be provided with the arm. In some embodiments, the rigid arm 316 includes a proximal end 318 including one or more through-holes 320 in alignment with the interbody spacer 314 to guide the instruments on the determined insertion trajectory.

The term “coupled” is defined as connected, although not necessarily directly, and not necessarily mechanically. The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way but may also be configured in ways that are not listed.

All patents and publications mentioned in this specification are indicative of the levels of those skilled in the art to which the disclosure pertains. It is to be understood that while a certain form of the disclosure is illustrated, it is not to be limited to the specific form or arrangement herein described and shown. It will be apparent to those skilled in the art that various changes may be made without departing from the scope of the disclosure and the disclosure is not to be considered limited to what is shown and described in the specification and any drawings/figures included herein.

One skilled in the art will readily appreciate that the present disclosure is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the disclosure and are defined by the scope of the appended claims. Although the disclosure has been described in connection with specific preferred embodiments, it should be understood that the disclosure as claimed should not be unduly limited to such specific embodiments. Indeed, various modifications of the described modes for carrying out the disclosure which are obvious to those skilled in the art are intended to be within the scope of the following claims.

What is claimed is:

1. A surgical fixation assembly, comprising:
a compression member comprising a cylindrical body having a proximal end, a distal end, a middle portion extending longitudinally between the proximal and distal ends, a first bone engaging feature positioned at the proximal end, a second bone engaging feature positioned at the distal end, and a stabilizer engagement feature positioned on an exterior surface of the compression member;
an independent stabilizer configured to couple to the compression member by interacting with the stabilizer engagement feature, the stabilizer comprising an elongated body having a third bone engagement feature and a coupling feature; and
a locking element rotatably coupled to the stabilizer, the locking element configured for movement between a first position enabling movement of the stabilizer relative to the compression member before or during coupling of the stabilizer and compression member, and a second position preventing movement of the stabilizer relative to the compression member after coupling of the stabilizer and compression member.

2. The assembly of claim 1, wherein the first bone engaging feature comprises a helical thread disposed about the exterior surface of the cylindrical body at the proximal end.

3. The assembly of claim 1, wherein the second bone engaging feature comprises a helical thread disposed about the exterior surface of the cylindrical body at the distal end.

4. The assembly of claim 1, wherein the stabilizer engagement feature comprises a longitudinal recess formed within the exterior surface of the cylindrical body and extending between the proximal and distal ends.

5. The assembly of claim 4, wherein the longitudinal recess is configured to slideably receive at least a portion of the stabilizer therein.

6. The assembly of claim 1, wherein the compression member has an inner lumen extending longitudinally through the elongated body between openings at the proximal and distal ends.

7. The assembly of claim 1, wherein the compression member includes at least one lateral opening formed within the elongated body.

8. The assembly of claim 1, wherein the stabilizer has a triangular cross-sectional shape.

9. The assembly of claim 1, wherein the stabilizer has at least one transverse opening formed therein.

10. The assembly of claim 1, wherein the locking element has at least one lateral flange configured to engage the compression member when the locking element is in the second position.

11. A method of fusing a first bone segment and a second bone segment across a joint between the first and second bone segments, comprising:
establishing an operative corridor to a surgical target site;
securing a compression member to a driver instrument having an elongated drill bit at a distal end, the compression member comprising a cylindrical body having a proximal end, a distal end, a middle portion extending longitudinally between the proximal and distal ends, a first bone engaging feature positioned at the proximal end, a second bone engaging feature positioned at the distal end, a stabilizer engagement feature positioned on an exterior surface of the compression member, and an inner lumen extending longitudinally through the elongated body between openings at the proximal and distal ends;
implanting the compression member into the surgical target site by rotatably advancing the compression member through the first bone segment, across the joint and into the second bone segment until the second bone engaging feature is seated within the second bone segment, the first bone engaging feature is seated within the first bone segment, and the middle portion extends across the joint;
advancing an independent stabilizer through the operative corridor toward the implanted compression member;
coupling the independent stabilizer in situ to the implanted compression member such that the implanted compression member is prevented from rotating relative to the first and second bone segments; and
upon in situ coupling of the stabilizer and the compression member, rotating a locking element coupled with the stabilizer from a first position enabling movement of the stabilizer relative to the compression member, to a second position preventing movement of the stabilizer relative to the compression member.

12. The method of claim 11, wherein the compression member is secured to the driver instrument such that a distal tip portion of the elongated drill bit extends through the inner lumen of the compression member and protrudes distally from the distal end of the compression member.

13. The method of claim 12, wherein the step of implanting the compression member within the surgical target site includes the sub-steps of:
advancing the driver instrument with secured compression member distally through the operative corridor until the distal tip portion of the drill bit contacts the first bone segment; and
operating the driver instrument such that the drill bit and compression member rotate.

14. The method of claim 12, wherein prior to performing the step of installing the stabilizer, the driver instrument is detached from the compression member and removed from the operative corridor.

15. The method of claim 11, wherein the first bone engaging feature comprises a helical thread disposed about the exterior surface of the cylindrical body at the proximal end.

16. The method of claim 11, wherein the second bone engaging feature comprises a helical thread disposed about the exterior surface of the cylindrical body at the distal end.

17. The method of claim 11, wherein the step of coupling the independent stabilizer in situ to the implanted compression member comprises the sub-steps of:
positioning a portion of a coupling element provided on the stabilizer into a portion of the stabilizer engagement feature of the compression member; and
advancing the stabilizer distally along the compression member by slideably translating the coupling element within the stabilizer engagement feature.

18. The method of claim 17, wherein the coupling element comprises an elongated guide rail and the stabilizer engagement feature comprises an elongated track.

19. The method of claim 18, wherein the elongated guide rail is secured within the elongated track with a dovetail feature.

20. The method of claim 11, wherein the locking element has at least one lateral flange configured to engage the compression member when the locking element is in the second position.
